(19) United States
(12) Patent Application Publication
(10) Pub. No.: US 2004/0133102 A1
(43) Pub. Date: Jul. 8, 2004

(54) Radiotherapeutic aid assembly and radiotherapeutic aid

(76) Inventor: Minoru Uematsu, Kamakura-shi (JP)

Correspondence Address: BIRCH STEWART KOLASCH & BIRCH, PO BOX 747, FALLS CHURCH, VA, 22040-0747, US

(21) Appl. No.: 10/737,834

(22) Filed: Dec. 18, 2003

(30) Foreign Application Priority Data
Jan. 6, 2003 (JP) 2003-000092

(51) Int. Cl. A61N 5/10

(52) U.S. Cl. 600/436; 378/208; 378/65

(57) ABSTRACT

A radiotherapeutic aid assembly for forming a radiotherapeutic aid for use in at least one of applications including marking an irradiation location in breast radiotherapy, fixing a patient, and assisting in stabilizing the shape of an affected part, comprises a first strip shell member, a second strip shell member, and a third strip shell member. The first through third strip shell members can be shaped along a body surface of the patient in surrounding relation to an irradiation area on the body surface.

### BACKGROUND OF THE INVENTION

1. Field of the Invention

The present invention relates to a radiotherapeutic aid assembly for use in breast radiotherapy, e.g., an irradiation area marking member capable of marking for irradiation positioning without the need for marking on the skin of the radiation recipient, a fixing member for fixing the patient in position on a treatment bed, or a shape stabilization aid assembly capable of stabilizing the shape of an area, which is easily deformable such as a breast, to be irradiated. More particularly, the present invention relates to an aid assembly suitable for use in therapy, such as an irradiation location marking member capable of marking an irradiation area that has been determined by a therapeutic plan, for example, and that is to be irradiated repeatedly with good positional reproducibility for a certain period of time for radiotherapy, a fixing member for producing a fixing device representing the shape of an easily deformable area, such as a breast, including an affected part of the patient, without deforming the area with shell members that are shaped along the area, the fixing member being capable of fixing the patient on a treatment bed with good positional reproducibility for repeated radiotherapeutic sessions, or a shape stabilization aid assembly capable of stabilizing the shape of an area, which is easily deformable such as a breast, to be irradiated.

The present invention is also concerned with a therapeutic aid for use as a marking outfit, a fixing device, or a shape stabilization aid for radiotherapy. More specifically, the present invention is concerned with a therapeutic aid which can be used as a marking outfit comprising an aid marked at necessary locations and having a shaped assembly of shell members shaped along the profile of an established irradiation area or a nearby body surface of the patient in surrounding relation to the irradiation area, the marking outfit being worn by the patient when it is repeatedly irradiated with a dose of radiation with as good positional reproducibility as if the skin of the patient is directly marked with markers, a fixing device for fixing the patient, or a shape stabilization aid for assisting in stabilizing the shape of the affected part of the patient.

2. Description of the Related Art

In radiotherapy, such as cancer radiotherapy, where the affected part such as a tumor region in a human body is irradiated with a dose of radiation, a therapeutic plan is prepared prior to the treatment. The therapeutic plan is produced by specifying an affected region, such as a tumor region, in the body based on medical images of the patient (radiation recipient) which is generated by an X-ray CT system or the like, and simulating therapeutic details with a planning system including a device for capturing collected image information and a computer system made up of a display unit, an input unit, a CPU (central processing unit), etc., to determine an optimum process of irradiating the specified affected region, i.e., the range to be irradiated with a dose of therapeutic radiation, the direction in which the dose of therapeutic radiation is applied, the intensity of the dose of therapeutic radiation, and the time for which the dose of therapeutic radiation is applied.

The therapeutic plan thus drawn up needs to be reflected accurately in a radiotherapeutic process carried out by a radiotherapeutic apparatus. Particularly, the position of the affected part which is to be irradiated is one of important elements to be considered in the radiotherapeutic process. For increasing the accuracy of the position to be irradiated, it is customary to mark the skin of the body surface of the radiation recipient to which a dose of therapeutic radiation is to be applied before it is irradiated with the dose of therapeutic radiation according to the therapeutic plan. Specifically, the operator uses an X-ray simulator, for example, to apply a laser beam to the body surface of the radiation recipient to position an irradiation field (irradiation range) thereon, and then mark the body surface (skin) at a location indicated by the laser beam, e.g., a profile or center of the irradiation field, directly with hardly erasable ink from a felt-tipped marker pen or an ordinary pen.

During the period of time (e.g., about one to two months) in which the affected part is to be repeatedly irradiated with a dose of radiation, the marking on the body surface cannot be erased because the affected part is repeatedly irradiated using the marking on the skin as a visual guide. In breast conservation therapy, for example, when an irradiation field is to be positioned on the patient or radiation recipient, the patient is stripped to the waist, and as shown in FIG. 3 of the accompanying drawings, and the operator uses a fluoroscopic system or the like to determine an appropriate irradiation field for applying a dose 20 of therapeutic radiation to both sides (see the arrows 20a, 20b) of an entire breast 10b of the radiation recipient 10 where a tumor 10a is present. To give a visual indication of the determined irradiation field, the operator marks the skin at locations 10c which correspond to the profile and center of the irradiation field. Subsequently, the patient who is stripped to the waist is irradiated repeatedly day by day using the marking as a visual aid. For details of breast conservation therapy, reference should be made to “Practical breast conservation therapy” edited by Hiroki Oyama, published by K.K. Medical Journal, Feb. 20, 1999, p. 34, figure. 1. FIG. 3 schematically shows breasts of the radiation recipient as viewed from its feet for illustrating an irradiation process in breast conservation therapy and a process of marking an irradiation area.

The above process is a standard radiotherapeutic process that is normally practiced all over the world at present. However, this process is inconvenient and mentally burdensome for the patient because the marking on the skin should be protected against accidental erasure for a long period of time, e.g., from one to two months. Furthermore, the process is also mentally painful for the patient as the affected part and nearby area have to be exposed to view everyday though the viewer is medical personnel.

There has been proposed a radiotherapeutic apparatus with an improved means for positioning the patient with respect to an irradiation area such that the marking can be erased after the first treatment (see Japanese Patent Laid-open No. Hei 10-24118). There has also been proposed a positioning sheet which is capable of positioning the patient with an irradiation apparatus without the need for marking the patient (see Japanese Patent Laid-open No. Hei 10-76020).

However, the proposed apparatus and sheet require complex apparatus and expensive equipment. It has thus been desired to develop a technique to irradiate the patient with high positional accuracy in the same manner as if the skin were marked, without the need for the installation of special apparatus and equipment and complex means.

It has heretofore been customary in radiotherapy to use a single shell blank to cover a wide body area including an affected part, and shape the shell blank into a shell which is to be used to cover the affected part. In head and neck cancer radiotherapy, it has been practiced to produce a fixture (shell) of vinyl chloride, apply markers to the surface of the shell at spaced intervals of about 2 cm, perform CT scanning on the patient who is wearing the shell, and draw an irradiation field on the surface of the shell using the markers as indicators based on the CT image that has been obtained. For details, see “Radiotherapy and CT”, edited by Tadayoshi Matsuda, published by K.K. Shuujunsha, Dec. 20, 1982, p. 48, left column, line 19 to line 25, FIGS. II-14, FIGS. II-15. When an affected part is widely covered with a shell, the shell tends to increase a dose of radiation applied to the skin. In order to reduce such a phenomenon, it has been proposed to use a mesh-type shell blank having a number of holes defined therein or to apply a shell blank closely to the shape of the overall face of the patient, perform radiational cooling of the shell blank to produce a shell of desired shape, and then define an opening in the shell for passage of a radiation therethrough (see Japanese Patent Laid-open No. 2001-170071 (FIG. 7)).

When a body region which is easily deformable such as a breast is covered with a shell blank to produce a shell therefrom, the breast is liable to be pressed and deformed by the shell blank. A shell that is produced from the shell blank thus applied is of a deformed shape. When the shell is mounted on the breast for irradiation, the breast is deformed by the shell, and the irradiation area possibly becomes inadequate. In addition, as described above, the shell increases the dose of radiation applied to the skin, which intensifies the skin reaction due to the radiotherapy, tending to impair the cosmetic effect. The mesh-type shell or the shell with the opening for passage of a radiation therethrough does not solve the problem that the affected part present in an easily deformable region tends to be pressed and deformed by the shell. Though the mesh-type shell is capable of slightly reducing the increase in the dose of radiation applied to the skin, it fails to eliminate the increase in the dose of radiation. In the case where the affected part is present in an easily deformable region, therapeutic accuracy can be increased if the irradiation area can be stabilized in shape during the treatment.

### BRIEF SUMMARY OF THE INVENTION

It is therefore an object of the present invention to provide a radiotherapeutic aid assembly for use as an irradiation area marking assembly capable of marking for irradiation positioning without the need for directly marking on the skin of the radiation recipient, without the need for erasing markers on the skis during a repeated irradiation period, and without increasing the dose of radiation applied to the skin, when a breast is irradiated with a dose of radiation in radiotherapy such as breast conservation therapy, a fixing device capable of fixing an easily deformable affected part such as a breast while minimizing any deformation of the affected part, or a shape stabilization aid assembly capable of stabilizing the shape of the affected part or breast in an irradiation area.

Another object of the present invention to provide a therapeutic aid for use as a marking outfit which is worn by the patient to allow the patient to be repeatedly irradiated with a dose of radiation with good positional reproducibility, a fixing device for fixing the patient in position while preventing an irradiation area on the patient from being deformed or displaced, or a shape stabilization aid capable of stabilizing the shape of the irradiation area.

A therapeutic aid assembly according to the present invention has at least three strip shell members (first, second, and third strip shell members) that can be shaped along the body surface of the patient in surrounding relation to an irradiation area on the body surface, thus forming a therapeutic aid for use at least one of applications including marking irradiation locations for therapy, fixing the patient in position, and assisting in stabilizing the shape of the affected part of the patient. The therapeutic aid assembly according to the present invention can be used as an irradiation location marking assembly of an entirely new concept which is produced by marking a shaped assembly shaped around an irradiation area with markings required to irradiate irradiation locations determined by a therapeutic plan, repeatedly with good positional reproducibility for radiotherapy. The therapeutic aid assembly can thus be useful as a marking assembly among other applications. The aid assembly according to the present invention can be used in any one of the applications including marking irradiation locations for therapy, fixing the patient in position, and assisting in stabilizing the shape of the affected part of the patient. The aid assembly may also be used simultaneously in two applications such as marking and fixing applications or marking and shape stabilization assisting applications, or simultaneously in all the three applications described above.

The aid assembly according to the present invention has a first strip shell member, a second strip shell member, and a third strip shell member, and the first through third strip shell members can be shaped along a body surface of the patient in surrounding relation to an irradiation area on the body surface. By placing these strip shell members on the body surface of the patient in surrounding relation to the irradiation area thereon, they can be formed as a shaped assembly shaped along the body surface of the patient in surrounding relation to the irradiation area. The three strip shell members are not required to surround the entire irradiation area, but the irradiation area may have an upper side, a lower side, and a right side surrounded by the three strip shell members, and may have a left side open. Furthermore, as described later, a fourth strip shell member may be added to the three strip shell members to surround the four sides of the irradiation area.

The strip shell members may be placed directly on the body surface of the patient or radiation recipient, or may be placed on a radiation-permeable thin clothing such as a T-shirt worn by the radiation recipient. The shaped assembly produced from the aid assembly may be placed on the body surface of the patient or worn by the patient, and marked with markings as descried above. The shaped assembly thus marked can be used as a radiotherapeutic aid which is used as a marking outfit in marking irradiation locations in radiotherapy. Alternatively, the shaped assembly produced from the aid assembly may be fixed to a treatment bed. The shaped assembly thus fixed can be used as a fixing device for fixing the patient or an affected part thereof in position. Further alternatively, the shaped assembly produced from the aid assembly may be placed around an irradiation area of the patient with a T-shirt, for example, covering the irradiation area. The shaped assembly thus placed can be used as a shape stabilization aid for use in assisting in stabilizing the shape of the affected part in the irradiation area.

The three strip shell members of the therapeutic aid assembly according to the present invention may be placed to surround the respective three sides of the irradiation area which is of a substantially square shape. Specifically, the first strip shell member can be shaped along the body surface so as to extend in a direction over the body of the patient lying on a treatment bed through a position near the irradiation area, the second strip shell member can be shaped along the body surface so as to extend in the direction through a position near the irradiation area opposite to the first strip shell member, at least one of the first strip shell member and the second strip shell member extending from one end to the other of the patient on the treatment bed, and the third strip shell member can be shaped along the body surface so as to extend through a position near the irradiation area between the first strip shell member and the second strip shell member. With the above arrangement, when the strip shell members are placed on the body surface of the patient put (usually lying) on the treatment bed at the specified regions, and shaped along the body surface at the specified regions, the strip shell members are shaped into a shaped assembly matching the shape of the body surface of the patient in surrounding relation to the irradiation area on its three sides, with the remaining one side being open. The shaped assembly having portions extending to both sides of the body of the patient.

The aid assembly according to the present invention may further include a fourth strip shell member which can be shaped along the body surface so as to extend through a position near the irradiation area opposite to the third strip shell member. The first through fourth strip shell members are shaped into a shaped assembly surrounding the irradiation area on its all four sides, and have portions extending to both sides of the body of the patient.

In a radiotherapeutic process where one irradiation area of the body of the patient is repeatedly irradiated by a radiation source positioned remotely from the patient, an irradiation or treatment bed capable of fixing the posture of the patient thereon is usually used to improve the positional reproducibility of the irradiated posture of the patient or radiation recipient which has been positioned. If at least one of the first strip shell member and the second strip shell member extending from one end to the other of the patient has fixing tabs for fixing the patient to the treatment bed, then the positional and postural reproducibility of the patient on the treatment bed is increased for repeated radiotherapeutic sessions.

If the aid assembly further has an X-ray impermeable irradiation area setting member, then it may be fixed to the shaped assembly and shaped to indicate an irradiation area in a therapeutic process using X-rays for irradiation. For example, the X-ray impermeable irradiation area setting member may be used as a reference line for positioning in fluoroscopy. With the aid assembly having the an X-ray impermeable irradiation area setting member, it may be attached to the first strip shell assembly, or may not be fixed to a strip shell assembly in advance, but may be attached thereto after being shaped.

If at least one of the first strip shell member and the second strip shell member has X-ray impermeable markers, then the positions of irradiation locations can easily be confirmed in fluoroscopy, for example. Since the markers are captured in MR images and CT images, a recollation of MR images and CT images can easily be performed by setting marker positions. The markers are useful when MR images and CT images need to be positionally aligned by image translation and rotation.

The shaped assembly produced from the aid assembly according to the present invention is shaped along the body surface on or near the profile of the irradiation area on its outer side in surrounding relation to the irradiation area. Even if an affected part of the patient is present in a body region, such as a breast, that is easily deformable when pressed, the shaped assembly does not press the affected part, and hence minimizes the possibility that the irradiation area will be displaced by deformation of the irradiated body region.

When a shaped assembly is formed from a radiotherapeutic aid assembly according to the present invention, there is produced a therapeutic aid comprising a shaped assembly having a frame or an open frame of shell members shaped along a body surface of a patient in surrounding relation to an irradiation area on the body surface. The shaped assembly of the therapeutic aid may have a frame or an open frame shaped along the body surface of the patient near the irradiation area. For example, the frame or the open frame may be shaped of shell members formed substantially as a frame or shell members incorporated in a frame mold. The therapeutic aid can be used in any one of the applications including a marking outfit, a fixing device for fixing a patient, and a shape stabilization aid for stabilizing the shape of the affected part of the patient. The therapeutic aid may also be used simultaneously in two applications such as a marking outfit and a fixing device or a marking outfit and a shape stabilization aid, or simultaneously in all the three applications described above.

The frame or open frame of the shaped assembly of the therapeutic aid is not limited to any particular shapes. The frame or open frame may be an angular frame such as a square frame, or a circular frame such as a round frame, or may be a substantially angular frame such as a substantially square frame with one side or a portion being open, or a substantially circular frame such as a substantially round frame, or the like. The shaped assembly of the therapeutic aid may be constructed of the frame or open frame only. However, if the therapeutic aid should not be displaced after it has been worn by the patient when it is used in any one of the applications including marking irradiation locations for therapy, fixing the patient in position, and assisting in stabilizing the shape of the affected part of the patient, then it may be necessary to fix the shaped assembly to the body of the patient or the treatment bed with tapes or the like each time the therapeutic aid is worn by the patient. In radiotherapy, the radiation recipient (patient) is normally fixed in position and posture to the irradiation bed. Therefore, it is preferable to mount the therapeutic aid on the patient for marking irradiation locations for irradiation and assisting in fixing the radiation recipient in position on the irradiation bed.

In the therapeutic aid according to the present invention, the shell members should preferably include a first strip shell member shaped along the body surface so as to extend in a direction over the body of the patient lying on a treatment bed through a position near the irradiation area, a second strip shell member shaped along the body surface so as to extend in the direction through a position near the irradiation area opposite to the first strip shell member, a third strip shell member shaped along the body surface so as to extend through a position near the irradiation area between the first strip shell member and the second strip shell member, at least one of the first strip shell member and the second strip shell member extending from one end to the other of the patient on the treatment bed, the third strip shell member being fixed to the first strip shell member and the second strip shell member.

If the therapeutic aid further includes an X-ray impermeable irradiation area setting member and the X-ray impermeable irradiation area setting member is shaped to indicate the irradiation area, then it allows the operator to confirm the irradiation area easily in radiotherapy. The shaped assembly of the therapeutic aid should preferably be constructed of strip shell members which are the same as those of the therapeutic aid assembly according to the present invention as described above. Therefore, it is preferable for the shaped assembly to have a fourth strip shell member which can be shaped along the body surface so as to extend through a position near the irradiation area opposite to the third strip shell member, the fourth strip shell member being fixed to the first strip shell member and the second strip shell member. If at least one of the first strip shell member and the second strip shell member is shaped so as to extend from one end to the other of the patient on the treatment bed, then fixing tabs should preferably be mounted on either one of the first strip shell member and the second strip shell member. The above markers may be provided on at least one of the first strip shell member and the second strip shell member.

The radiotherapeutic aid according to the present invention may be placed on the body surface of the patient or worn by the patient, and the shaped assembly may be marked with markings as descried above. The shaped assembly thus marked can be used as a marking outfit in marking irradiation locations in radiotherapy. Alternatively, the shaped assembly may be fixed to a treatment bed. The shaped assembly thus fixed can be used as a fixing device for fixing the patient or an affected part thereof in position. Further alternatively, the shaped assembly may be placed around an irradiation area of the patient with a T-shirt, for example, covering the irradiation area. The shaped assembly thus placed can be used as a shape stabilization aid for use in assisting in stabilizing the shape of the affected part in the irradiation area. Since the radiotherapeutic aid according to the present invention is shaped in close contact with the body surface of the patient in a particular body region, the radiotherapeutic aid may be placed or worn repeatedly with good positional accuracy so as to well match the shape of the body surface. The radiotherapeutic aid according to the present invention is thus useful as a radiotherapeutic aid for increasing the reproducibility of the irradiated posture of the radiation recipient which has been positioned so as to be repeatedly irradiated by a radiation source positioned remotely from the patient. If the radiotherapeutic aid is used as a marking outfit for marking irradiation locations, for example, then when the radiotherapeutic aid is worn by the patient to match the body surface and the shaped assembly thereof is marked with all markings necessary to determine an irradiation area according to a therapeutic plan, the irradiation area can be marked according to the therapeutic plan as if the skin of the patient were directly marked. Consequently, a radiotherapeutic process can be performed to irradiate irradiation locations according to the therapeutic plan repeatedly with good positional reproducibility for a certain period of time without the need for directly marking the skin of the patient.

As described above, the shaped assembly of the radiotherapeutic aid according to the present invention is constructed of shell members shaped along the body surface of the patient around the irradiation area, and is worn by the patient so as to surround the irradiation area including the affected part. The shaped assembly worn by the patient during a radiotherapeutic process does not cover the irradiation area. Therefore, the dose of radiation applied to the skin is not increased by shell members in radiotherapy, and the radiotherapeutic aid does not obstruct the treatment. If the shaped assembly is constructed of shell members shaped along the body surface wearing thereon a thin clothing such as a T-shirt which is made of a material that does not obstruct the treatment, e.g., a radiation-permeable material, then the shaped assembly may be used as a marking outfit for making irradiation locations, for example, allowing the patient to be treated, e.g., irradiated with a dose of radiation, while wearing the clothing. Using the thin clothing that does not obstruct the treatment, the shaped assembly can be used as a shape stabilization aid for stabilizing the shape of an easily deformable body region such as a breast.

If the shaped assembly of the aid further has an X-ray impermeable irradiation area setting member and the X-ray impermeable irradiation area setting member is shaped to indicate the irradiation area, then it may be used as a reference line for positioning in fluoroscopy in a therapeutic process using X-rays for irradiation.

If the therapeutic aid is used in radiotherapy, then when the therapeutic aid is worn by the patient to match the body surface and the shaped assembly thereof is marked with all markings necessary to determine an irradiation area according to a therapeutic plan, the therapeutic aid serves as a marked outfit. When the patient wears the marked outfit so as to match the body surface in each of repeated therapeutic sessions, the skin of the patient does not need to be directly marked, and hence no attempts need to be made to keep any skin markings unremoved during the overall irradiation period. Consequently, it is possible to perform a radiotherapeutic process for irradiating irradiation locations determined according to the therapeutic plan, repeatedly with good positional reproducibility for a certain period of time.

The above and other objects, features, and advantages of the present invention will become more apparent from the following description when taken in conjunction with the accompanying drawings in which a preferred embodiment of the present invention is shown by way of illustrative example.

### BRIEF DESCRIPTION OF THE SEVERAL VIEWS OF THE DRAWINGS

FIG. 1 is a plan view of a radiotherapeutic aid assembly for use as an irradiation location marking assembly according to an embodiment of the present invention;

FIG. 2 is a plan view of a radiation recipient who is wearing a marking outfit as a radiotherapeutic aid according to the present invention; and

FIG. 3 is a diagram showing a dose of radiation applied to a radiation recipient, the diagram being illustrative of a process of marking irradiation locations in breast conservation therapy.

### DETAILED DESCRIPTION OF THE INVENTION

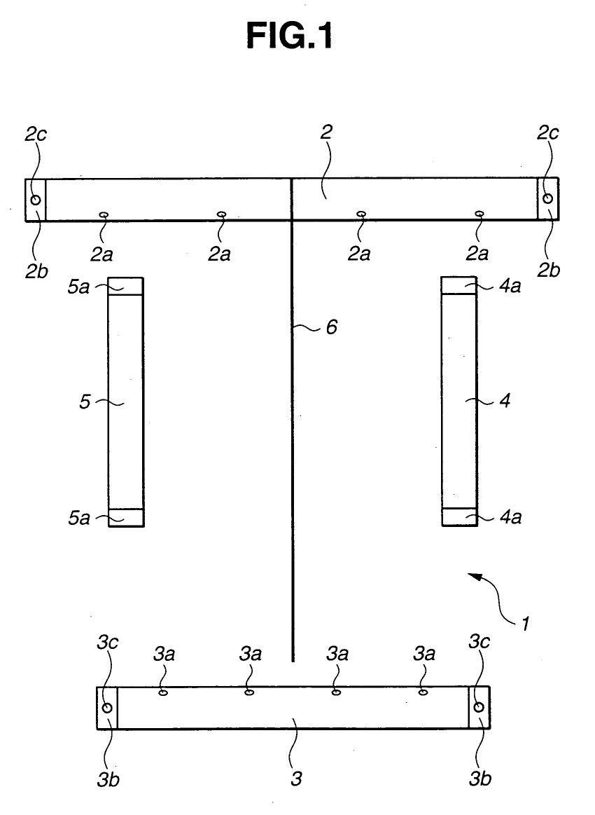

FIG. 1 shows in plan an aid assembly for use in radiotherapy as one form of an aid assembly for use in therapy according to an embodiment of the present invention. In FIG. 1, the aid assembly, generally denoted at 1, according to the embodiment of the present invention is used in breast radiotherapy such as breast conservation radiotherapy, and is primarily used in assisting in marking irradiation locations on the body of a patient or a radiation recipient. The aid assembly 1 includes a first strip shell member 2, a second strip shell member 3, a third strip shell member 4, and a fourth strip shell member 5. The first strip shell member 2 and the second strip shell member 3 are placed and shaped along the body surface of the patient transversely across the body of the patient at respective positions above and below an irradiation area of the patient who is lying on a treatment bed (not shown). The third strip shell member 4 and the fourth strip shell member 5 are placed and shaped along the body surface of the patient at respective positions leftward and rightward of the irradiation area of the patient across the first strip shell member 2 and the second strip shell member 3.

A plurality of (four in FIG. 1) X-ray impermeable markers 2a are fixed at spaced intervals to the first strip shell member 2 along a longitudinal edge thereof. An irradiation area setting member 6 for use in radiotherapy is fixed at an end thereof to a longitudinally central portion of the first strip shell member 2, and extends substantially perpendicularly to the first strip shell member 2. Fixing tabs 2b for fixing the first strip shell member 2 to the treatment bed which serves as the treatment bed (not shown) are formed on respective opposite longitudinal ends of the first strip shell member 2. According to the present invention, the positions and the number of markers are not limited, but two to ten markers, particularly four to six markers, should preferably be provided at spaced intervals along a longitudinal edge of the strip shell member for the ease with which to recognize the markers as indicators.

A plurality of (four in FIG. 1) X-ray impermeable markers 3a are fixed at spaced intervals to the second strip shell member 3 along a longitudinal edge thereof. Fixing tabs 3b for fixing the second strip shell member 3 to the treatment bed (not shown) are formed on respective opposite longitudinal ends of the second strip shell member 3. With the radiotherapeutic aid assembly according to the present invention, fixing tabs may be formed on either one of the first and second strip shell members. For better positional and postural reproducibility of the radiation recipient, however, it is more effective to provide fixing tabs on both the first and second strip shell members. Moreover, with the radiotherapeutic aid assembly having the X-ray impermeable markers, fixing tabs may be formed on either one of the first and second strip shell members, but it is more effective to provide fixing tabs on both the first and second strip shell members.

The third strip shell member 4 has grips 4a formed on respective opposite longitudinal ends thereof for allowing the third strip shell member 4 to be handled with ease when the third strip shell member 4 is in a loose state. Similarly, the fourth strip shell member 5 has grips 5a formed on respective opposite longitudinal ends thereof. The radiotherapeutic aid assembly according to the present invention may be free of the fourth strip shell member 5. However, for higher positional accuracy and better marking capability, it is preferable to have the fourth strip shell member 5 positioned across the irradiation area from the third strip shell member 4 and extending in the same direction as the third strip shell member 4.

The strip shell members 2, 3, 4 and 5 may be made of any of various materials. For example, the strip shell members may be made of a known shell material similar to the material of shells that are presently used as fixtures in radiotherapy. The strip shell members may be made of a thermoplastic material having a low melting point, which is softened and can greatly be stretched when heated to about 50 to 80° C., preferably 50 to 70° C. or more preferably 60 to 70° C., and which is hardened at normal temperature (skin surface temperature). For example, the strip shell members may be in the form of a plastic sheet having a thickness ranging from 1 to 3 mm and made of a thermoplastic material such as polyvinyl chloride, polyester, polyisoprene, polycaprolactone, or the like. Such a plastic sheet is commercially available as Shell Fitter, Orfit, Posicast, Uniframe, Thermoshell (tradename, manufactured by Alcare Co., Ltd. polycaprolactone). Alternatively, the strip shell members may be made of a material whose surface is treated with polyurethane, i.e., a mesh-type shell material (e.g., Thermoshell with holes (tradename, manufactured by Alcare Co., Ltd. polycaprolactone) or Thermoshell (tradename, manufactured by Alcare Co., Ltd. polycaprolactone sheet) on which polyurethane film is laminated. Furthermore, the strip shell members may be made of fatty polyester whose molecular weight ranges from 50 to 100 thousands, as disclosed in Japanese Patent Laid-open No. 2001-170071 and Japanese Patent Laid-open No. 2001-170073.

The strip shell members are obtained by cutting off any of the above commercially available thermoplastic sheets to suitable length and width, which are not limited to particular values. For example, the strip shell members as they are heated and softened may be of such lengths as to surround the irradiation area determined by the therapeutic plan and placed and shaped along the body surface of the patient. For example, in view of the accuracy with which the strip shell members are shaped along the body surface of the patient, the handling of the strip shell members, and the economy of the strip shell members, the first and second strip shell members 2, 3 may be of such dimensions as to be placed based on the standard widths of the bodies of females above and below the breasts. Specifically, for example, the first strip shell member 2 should preferably have a length ranging from 50 to 200 cm, particularly 70 to 150 cm and a width ranging from 1 to 10 cm, particularly from 3 to 7 cm, and the second strip shell member 3 should preferably have a length ranging from 30 to 200 cm, particularly from 50 to 150 cm, and a width ranging from 1 to 10 cm, particularly from 3 to 7 cm. The third and fourth strip shell members 4, 5 may be of such dimensions as to be placed based on the standard vertical lengths of the breasts of females. Specifically, each of the third and fourth strip shell members 4, 5 should preferably have a length ranging from 10 to 30 cm, particularly from 15 to 25 cm, and a width ranging from 1 to 10 cm, particularly from 3 to 7 cm.

There has been proposed a technique to put a material where the surface of small beads of foamed polystyrene is coated with an adhesive which will be solidified by added water, into a net of cloth having a suitable size, and adding water to produce a desired pillow or a main body portion holder within about 10 minutes. The material thus produced may be shaped into a strip and used as a strip shell member.

The fixing tabs 2b, 3b on the first strip shell member 2 and second strip shell member 3 may be selected depending on how they are fixed to the radiation treatment bed (not shown). For example, the fixing tabs 2b, 3b may include bands with a surface fastener, and may be fixed to the irradiation bed at predetermined positions thereon. In breast conservation therapy, for example, since a bed having fixtures for stably lifting upper both limbs or a limb near the affected part of the radiation recipient is generally used for positional reproducibility and postural reproducibility of the radiation recipient, the fixtures may include fitting or engaging portions, and rigid resin sheets may be boded to the opposite ends of the strip shell members by melting the opposite ends with heat or a solvent or by an adhesive, thus providing the fixing tabs 2b, 3b. Alternatively, the portions of the strip shell members which will serve as the fixing tabs 2b, 3b may be constructed of rigid resin sheets or may be hardened by suitable means into fitted or engaged portions (indicated by 2c, 3c in FIG. 1) matching the fitting or engaging portions. Further alternatively, the fixing tabs 2b, 3b themselves may be fitted in or over the opposite ends of the strip shell members.

The markers 2a, 3a fixed to the first and second strip shell members 2, 3 are not limited to any materials and shapes insofar as they are made of an X-ray impermeable material. For example, the markers 2a, 3a may be made of a metal such as lead, iron, aluminum, titanium, or the like, or ceramics, or an X-ray impermeable resin such as polytetrafluoroethylene, silicone, etc. Specifically, the markers 2a, 3a may be in the form of rubber erasures, a resin clips, metal clips, or the like.

The grips 4a, 5a of the third and fourth strip shell members 4, 5 are not limited to any materials and shapes insofar as they can easily be handled while they are warm. If the ends of the third and fourth strip shell members 4, 5 are backed up with a rigid resin material or nonwoven cloth, or the grips 4a, 5a are made of a rigid resin material or nonwoven cloth, then the ends of the third and fourth strip shell members 4, 5 or the grips 4a, 5a are prevented from being stretched when third and fourth strip shell members 4, 5 are warmed.

The irradiation area setting member 6 may be in the form of a member that can easily be bent to a desired shape and kept in the bent shape. Specifically, such a member may include a wire of metal such as lead, iron, aluminum, copper, titanium, or the like, or a thin sheet of metal such as aluminum or the like which is shaped into a thin slender member that can easily be bent for better formability. According to the present invention, the irradiation area setting member 6 may be fixed at its distal end to the first strip shell member 2 in advance or may be mounted on the first strip shell member 2 after it has been formed to shape. The irradiation area setting member 6 is not limited to any length, but may be of such a length as to be able to indicate a profile of the irradiation area or a region near the profile. For example, if the irradiation area setting member 6 is too long to surround substantially the entire profile of the irradiation area, then it may be cut off after it has been bent to shape.

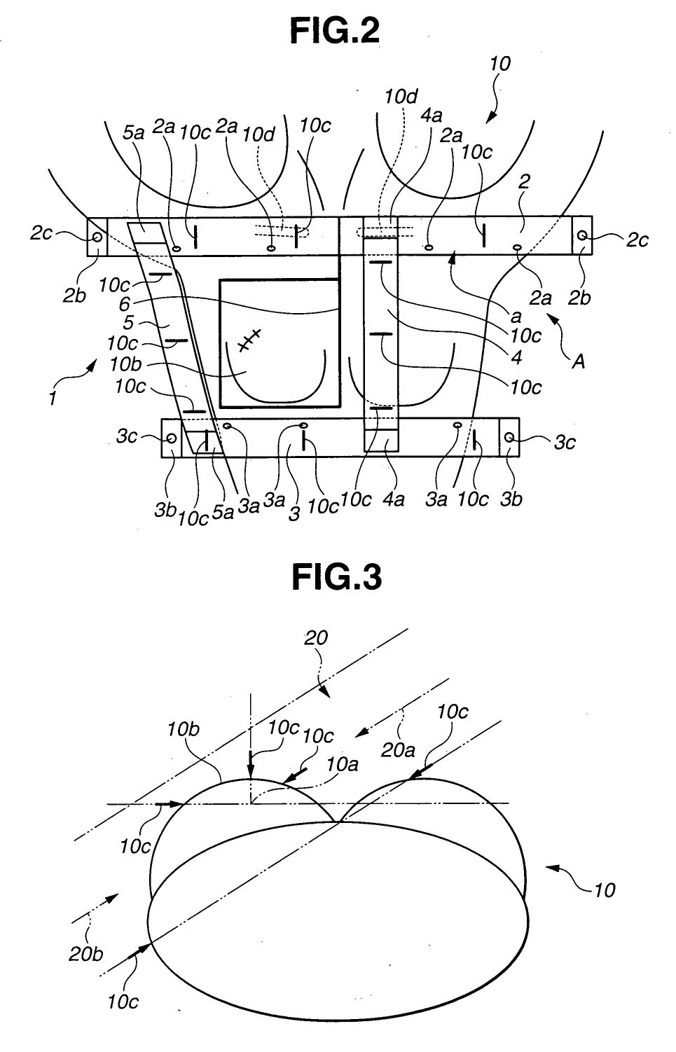

The aid assembly (irradiation location marking assembly) 1 operates as follows: The first strip shell member 2 is placed on the radiation recipient (patient) 10 (see FIG. 2) lying on the irradiation bed (not shown) substantially perpendicular to the body axis of the radiation recipient 10, i.e., transversely across the body of the radiation recipient 10. The second strip shell member 3 is similarly placed on the radiation recipient 10 transversely across the body of the radiation recipient 10 and substantially parallel to the first strip shell member 2. The third strip shell member 4 is placed on the radiation recipient 10 substantially parallel to the body axis thereof, i.e., longitudinally of the body of the radiation recipient 10. The fourth strip shell member 5 is placed on the radiation recipient 10 substantially in the same direction as the third strip shell member 4 and substantially parallel or slightly obliquely to the body axis of the radiation recipient 10. The first, second, third, and fourth strip shell members 2, 3, 4, 5 thus placed are shaped along the body surface of the radiation recipient 10 in surrounding relation to an irradiation area thereof.

A process of forming a radiotherapeutic aid using the aid assembly 1 will be described in detail below with reference to FIG. 2. FIG. 2 shows a radiotherapeutic aid formed from the aid assembly 1 and worn by the radiation recipient 10. Specifically, the radiotherapeutic aid is chiefly used as a marking outfit to be worn by the radiation recipient in breast conservation therapy.

When a shaped assembly “a” of a radiotherapeutic aid (marking outfit) A is to be formed, the radiation recipient 10 lies on an irradiation bed (not shown) with both upper limbs or one upper limb thereof (both upper limbs in FIG. 2) raised. According to the present invention, the radiation recipient 10 may wear a thin clothing, such as a T-shirt or the like, which does not obstruct a dose of irradiation applied thereto. If a T-shirt or the like is worn by the radiation recipient 10, it may effectively be used to stabilize the shape of the breasts of the radiation recipient 10.

The first, second, third, and fourth strip shell members 2, 3, 4, 5 are softened by being heated by hot water, a constant-temperature bath, a hot plate, or the like to a temperature at which they can be placed along the body surface of the radiation recipient 10, e.g., a temperature ranging from 50 to 70° C. or preferably a temperature ranging from 60 to 70° C. The first, second, third, and fourth strip shell members 2, 3, 4, 5 are then placed and spread on the skin of the radiation recipient 10 at respective positions. Then, if necessary, the first, second, third, and fourth strip shell members 2, 3, 4, 5 are softened again by hot air discharged from a drier or the like, and pressed against the body surface of the radiation recipient 10 so as to be shaped along the surface of the skin. At this time, the fixing tabs 2b, 3b of the first and second strip shell members 2, 3 should preferably be fixed to the irradiation bed at respective positions thereon. The first, second, third, and fourth strip shell members 2, 3, 4, 5 may not be heated in advance, but may first be placed on the body surface of the radiation recipient 10 and thereafter may be softened by a drier or the like into close contact with the body surface.

Locations where the first, second, third, and fourth strip shell members 2, 3, 4, 5 are placed along the body surface of the radiation recipient 10 will be described in detail below. First, the adequacy of a posture of the radiation recipient 10 which is suitable for an irradiation process expected in fluoroscopy for the radiation recipient 10 is confirmed. Then, the heated first strip shell member 2 is placed on the body of the radiation recipient 10 perpendicularly to the body axis, i.e., transversely across the body of the radiation recipient 10, such that the lower edge of the heated first strip shell member 2 where the markers 2a are positioned is oriented as a lower edge and extends over the centers of the humeral capitulums in fluoroscopy. The first strip shell member 2 is lightly pressed against the body surface so as to be shaped along the body surface, for memorizing the shape of the clavicles 10d, and the fixing tabs 2b are fastened to the irradiation bed.

Then, the irradiation area setting member 6 is lowered along the median line on the sternum, bent toward the affected part at the lower edge of the breast 10b of the patient, further bent along the central axillary line, and further bent toward the median line at the lower edge of the humeral capitulum. In this manner, the irradiation area setting member 6 is shaped into a substantially square shape in surrounding relation to the breast 10b including the affected part, near the profile of the irradiation area (near the inside edge of the profile in FIG. 2). The shape formed by the irradiation area setting member 6 may be located on or near the profile of the irradiation area, preferably near the profile of the irradiation area or the inside edge of the profile. It is preferable for the profile of the irradiation area to be spaced about 0 to +20 mm, preferably 0 to 20 mm, more preferably 0 to 10 mm, or further preferably 0 to 5 mm, outwardly of an area usually surrounded by any of various shapes including a square shape, an elliptical shape, a circular shape, etc. If the irradiation area setting member 6 is too long, its terminal end (free end) may be cut off at the position where it contacts the proximal end of the irradiation area setting member 6. The shape (square shape in FIG. 2) surrounded by the irradiation area setting member 6 may be used as a reference line for positioning in fluoroscopy, and the irradiation area setting member 6 may be removed upon irradiation.

The heated second strip shell member 3 is fixed in position in wrapping relation to the body of the patient so as to memorize the shape of costal bones such that the edge of the heated second strip shell member 3 where the markers 3a are bonded is oriented as an upper edge and spaced 5 to 20 mm from the lower edge of the breast 10b toward the feet. The heated third strip shell member 4 is stretched while the grips 4a are being held by hand, and spaced 5 to 20 mm from the median line on the sternum toward the unaffected breast. The heated third strip shell member 4 is positioned with its longitudinally opposite ends or the grips 4a being placed respectively on the surfaces of the first and second strip shell members 2, 3. The heated fourth strip shell member 5 is similarly stretched while the grips 5a are being held by hand, and spaced 5 to 20 mm from the central axillary line toward the back of the body. The heated fourth strip shell member 5 is positioned with its longitudinally opposite ends or the grips 4a being placed respectively on the surfaces of the first and second strip shell members 2, 3. In FIG. 2, the fourth strip shell member 5 is shown laterally of the body of the radiation recipient 10 for illustrative purposes, but is actually placed on the side surface of the body, not shown in FIG. 2, that extends 5 to 20 mm from the central axillary line toward the back of the body.

While the first through fourth strip shell members 2, 3, 4, 5 are being placed on the body of the radiation recipient 10, their temperature is gradually lowered with time, and hence the softened first through fourth strip shell members 2, 3, 4, 5 are gradually hardened to shape. The longitudinal opposite ends of the third and fourth strip shell members 4, 5 are then fixed by a fixing means such as adhesive bonding to the first and second strip shell members 2, 3. The fixing means is not limited to any specific forms, but may be the sticky nature of the strip shell members or an spray of an adhesive or a liquid adhesive which is capable of bonding the strip shell members to each other. Thereafter, the first through fourth strip shell members 2, 3, 4, 5 thus shaped and bonded together are removed from the body surface as a radiotherapeutic aid A having a shaped assembly “a” which is shaped neatly along the body surface of the radiation recipient 10 near the irradiation area.

It is preferable for accurate irradiation to maintain the posture, thus achieved, of the radiation recipient 10, perform CT or fluoroscopy based on the reference line provided as described above, determine an irradiation process, and confirm the appropriateness of the dose distribution. If the radiotherapeutic aid A is used as a marking outfit, then since the established irradiation field (irradiation area) is projected onto the skin surface of the patient by a laser beam, the irradiation field is traced and all the necessary lines required for irradiation in the irradiation field are marked as markers 10c on the surface of the shaped assembly “a” with a felt-tipped marker pen or the like, thus performing the marking necessary for irradiation. The positioning of the radiation recipient 10 with a clothing on is now finished. The radiotherapeutic aid A with the shaped assembly “a” marked with all the markers 10c necessary for determining the irradiation area can be used as a marking outfit. The radiation recipient 10 who is wearing the marked radiotherapeutic aid A on its clothing, such as a T-shirt or the like, can be irradiated with a dose of radiation without exposing the skin to view. When the radiation recipient 10 is to be irradiated, the radiation recipient 10 wears the marked radiotherapeutic aid A that is customized to the body shape of the radiation recipient 10. The position to be irradiated is confirmed based on the markers 10c on the radiotherapeutic aid A, and the radiation recipient 10 is irradiated. The radiation recipient 10 can thus be irradiated with the clothing on.

The radiotherapeutic aid A may be used in another application. Specifically, the radiation recipient 10 lies on the irradiation bed in a predetermined posture with both limbs or a limb near the affected part being lifted and fixed by the fixtures that are normally used on the irradiation bed. Then, the shaped assembly “a” is formed with the fixing tabs 2b, 3b of the first and second strip shell members 2, 3 being fastened to the irradiation bed. For positioning and irradiating the radiation recipient 10, the radiation recipient 10 on the irradiation bed wears the radiotherapeutic aid A, and the radiotherapeutic aid A is fastened to the irradiation bed by the fixing tabs 2b, 3b. Thus, both limbs or a limb near the affected part of the radiation recipient 10 is fixed in position by the normal fixtures, and the body (chest) of the radiation recipient 10 is also fixed in position by the radiotherapeutic aid A. Therefore, the positional reproducibility and postural reproducibility of the radiation recipient 10 can be increased during a radiotherapeutic session. Consequently, the radiotherapeutic aid A can be used as a fixing device for fixing the radiation recipient 10 in position on the irradiation bed.

When the shaped assembly “a” shaped along the body surface of the radiation recipient 10 who is wearing a T-shirt or the like is used, and the radiotherapeutic aid A is mounted on the radiation recipient 10 who is wearing a similar T-shirt upon positioning or irradiation, the body area around the breasts of the radiation recipient 10 is framed by the shaping assembly “a”, and the breasts are covered with the T-shirt in the frame of the shaped assembly “a”. Therefore, the shape of the breasts that are easily deformable is stabilized. The radiotherapeutic aid A can thus be used as a shape stabilization aid for stabilizing the shape of a body region including the affected part, e.g., a breast in the illustrated embodiment.

The size and shape of each of the strip shell members can be selected as desired depending on the purpose for which the radiotherapeutic aid is to be used, e.g., a marking aid, a fixing aid, a shape stabilization aid, or the like.

According to the present invention, it is possible to mark for irradiation positioning, fix the patient and the affect part, or assist in stabilizing the shape of the affect part during the radiotherapy, without deforming the breast including the affected part, and without increasing a dose of radiation applied to the skin in breast radiotherapy. Furthermore, for example, it is possible to receive accurate radiotherapy even if the patient wears a T-shirt or the like.

Although a certain preferred embodiment of the present invention has been shown and described in detail, it should be understood that various changes and modifications may be made therein without departing from the scope of the appended claims.

What is claimed is:

1. A radiotherapeutic aid assembly for forming a radiotherapeutic aid for use in at least one of applications including marking an irradiation location in breast radiotherapy, fixing a patient, and assisting in stabilizing the shape of an affected part, comprising a first strip shell member, a second strip shell member, and a third strip shell member, wherein said first through third strip shell members can be shaped along a body surface of the patient in surrounding relation to an irradiation area on the body surface.

2. A radiotherapeutic aid assembly according to claim 1, wherein said first strip shell member can be shaped along the body surface so as to extend in a direction over the body of the patient lying on a treatment bed through a position near said irradiation area, said second strip shell member can be shaped along the body surface so as to extend in said direction through a position near said irradiation area opposite to said first strip shell member, at least one of said first strip shell member and said second strip shell member extending from one end to the other of the patient body on said treatment bed, and wherein said third strip shell member can be shaped along the body surface of the patient so as to extend through a position near said irradiation area between said first strip shell member and said second strip shell member.

3. A radiotherapeutic aid assembly according to claim 1, further comprising a fourth strip shell member which can be shaped along the body surface of the patient so as to extend through a position near said irradiation area opposite to said third strip shell member.

4. A radiotherapeutic aid assembly according to claim 1, wherein at least one of said first strip shell member and said second strip shell member has fixing tabs for fixing the patient to the treatment bed.

5. A radiotherapeutic aid assembly according to claim 1, further comprising an X-ray impermeable irradiation area setting member.

6. A radiotherapeutic aid assembly according to claim 1, wherein at least one of said first strip shell member and said second strip shell member has X-ray impermeable markers.

7. A therapeutic aid for use in at least one of applications including a marking outfit in breast radiotherapy, a fixing device, and a shape stabilization aid, comprising a shaped assembly having a frame or an open frame of shell members shaped along a body surface of a patient in surrounding relation to an irradiation area on the body surface.

8. A therapeutic aid according to claim 7, wherein said shell members include a first strip shell member shaped along the body surface so as to extend in a direction over the body of the patient lying on a treatment bed through a position near said irradiation area, a second strip shell member shaped along the body surface so as to extend in said direction through a position near said irradiation area opposite to said first strip shell member, a third strip shell member shaped along the body surface so as to extend through a position near said irradiation area between said first strip shell member and said second strip shell member, at least one of said first strip shell member and said second strip shell member extending from one end to the other of the patient body on said treatment bed, said third strip shell member being fixed to said first strip shell member and said second strip shell member.

9. A therapeutic aid according to claim 7, further comprising an X-ray impermeable irradiation area setting member, said X-ray impermeable irradiation area setting member being shaped to indicate said irradiation area.
